(19) United States
(12) Patent Application Publication
(10) Pub. No.: US 2026/0165771 A1
(43) Pub. Date: Jun. 18, 2026

(54) Electrosurgical Device with Integrated Smoke Evacuation and Filter Features

(71) Applicant: Stryker European Operations Limited, Cork (IE)

(72) Inventors: Micheal Burke, Cork (IE); Paul Sheridan, Wexford (IE); Stephen Faul, Cork (IE)

(21) Appl. No.: 19/129,231

(22) PCT Filed: Nov. 13, 2023
(86) PCT No.: PCT/IB2023/000687

§ 371 Date: May 12, 2025

Related U.S. Application Data
(60) Provisional application No. 63/425,352, filed on Nov. 15, 2022.

(51) Int. Cl. A61B 18/14; A61B 18/00

(52) U.S. Cl. CPC A61B 18/1402; A61B 2018/00642; A61B 2018/00744; A61B 2560/0266

(57) ABSTRACT

An example electrosurgical device includes: a housing defining therein a smoke flow path for evacuating surgical smoke; an electrosurgical electrode extending from the housing; an electric motor; a fan coupled to the electric motor; and a filter disposed along the smoke flow path within the housing, wherein as the electric motor rotates the fan, the fan draws the surgical smoke to flow within the housing through the filter, then flow to an external environment of the electrosurgical device.

### CROSS-REFERENCE TO RELATED APPLICATION

This application claims priority to U.S. Provisional Patent Application No. 63/425,352 filed Nov. 15, 2022, the entire contents of which are incorporated herein by reference.

### FIELD

The present disclosure generally relates to electrosurgical devices and, more specifically, to electrosurgical devices with an integrated system for surgical smoke filtration.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”). As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue).

Smoke is generated because of such tissue modification, and such smoke can be toxic. Conventional systems have a separate pump and a separate smoke tube that can be used to evacuate the smoke during electrosurgery. Such configuration increases cost and complexity of an electrosurgery system and may increase strain on a surgeon's hand. It is with respect to these and other considerations that the disclosure made herein is presented.

### SUMMARY

Within examples described herein, systems and methods for an electrosurgical device with integrated smoke evacuation and filter features.

Within additional examples described herein, systems and methods are described that relate to a surgical smoke evacuation and filtration system embedded within or coupled to an electrosurgical device and including an electric motor, a fan drivable by the electric motor, and a filter. As the electric motor drives the fan, the fan causes the surgical smoke to flow through the filter to rid the smoke of toxic materials or particles. In an example, the filter can also disinfect the smoke. Filtered smoke (i.e., the cleaned air) can then be discharged from the electrosurgical device.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying Figures.

FIG. 1 illustrates an electrosurgical system having an electrosurgical generator and an electrosurgical device, in accordance with an example implementation.

FIG. 2 illustrates a perspective view of an electrosurgical device, in accordance with an example implementation.

FIG. 3 illustrates a partial view of an electrosurgical device with a smoke evacuation and filtration system, in accordance with an example implementation.

FIG. 4 illustrates a smoke evacuation and filtration system, in accordance with an example implementation.

FIG. 5A illustrates a perspective view of a smoke evacuation and filtration system having a filter housing, in accordance with an example implementation.

FIG. 5B illustrates a partial perspective view of the smoke evacuation and filtration system of FIG. 5A showing an electric motor and a fan, in accordance with an example implementation.

FIG. 5C illustrates another partial perspective view of the smoke evacuation and filtration system of FIG. 5A showing the electric motor, the fan, and a collar, in accordance with an example implementation.

FIG. 5D illustrates another partial perspective view of the smoke evacuation and filtration system of FIG. 5A showing the filter without the filter housing, in accordance with an example implementation.

FIG. 6A illustrates a partial perspective view of an electrosurgical device, in accordance with an example implementation.

FIG. 6B illustrates a partial perspective view of the electrosurgical device of FIG. 6A from a different angle, in accordance with an example implementation.

FIG. 7 is a flowchart of a method of forming an electrosurgical device, in accordance with an example implementation.

FIG. 8 is a flowchart of a method of operating an electrosurgical device, in accordance with an example implementation.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

An electrosurgical device (e.g., an electrosurgical tool such as an electrosurgical pencil) can use electrical energy supplied by an electrosurgical generator to apply electrosurgical energy from an electrosurgical electrode to a tissue. Toxic fumes or smoke is generated when electrosurgical energy is applied to the tissue. Conventional systems include a separate vacuum source (e.g., a pump) and a smoke tube that is coupled to the electrosurgical device to evacuate the smoke away from the operating room. However, such implementation is costly and complex, and may cause strain on a surgeon's hand.

Disclosed herein are systems for eliminating such external vacuum source and smoke tube. Particularly, the disclosed systems include a smoke evacuation filtration assembly/system of an electric motor, a fan or impeller, and a filter that are integrated within or coupled to the electrosurgical device. As the electric motor is actuated, the electric motor drives the fan to suction the smoke and draw the smoke through the filter to rid the smoke of toxic components. In an example, the filter an also include a disinfectant to rid the smoke of bacteria or other harmful organisms. The filtered and disinfected smoke can then be released safely to the operating environment.

In one example, the operator can trigger the electric motor via an input device (e.g., a button). In another example, the system may additionally include a sensor to detect smoke and/or the amount of smoke being generated, and may automatically operate the motor at a particular speed to operate the smoke filtration system.

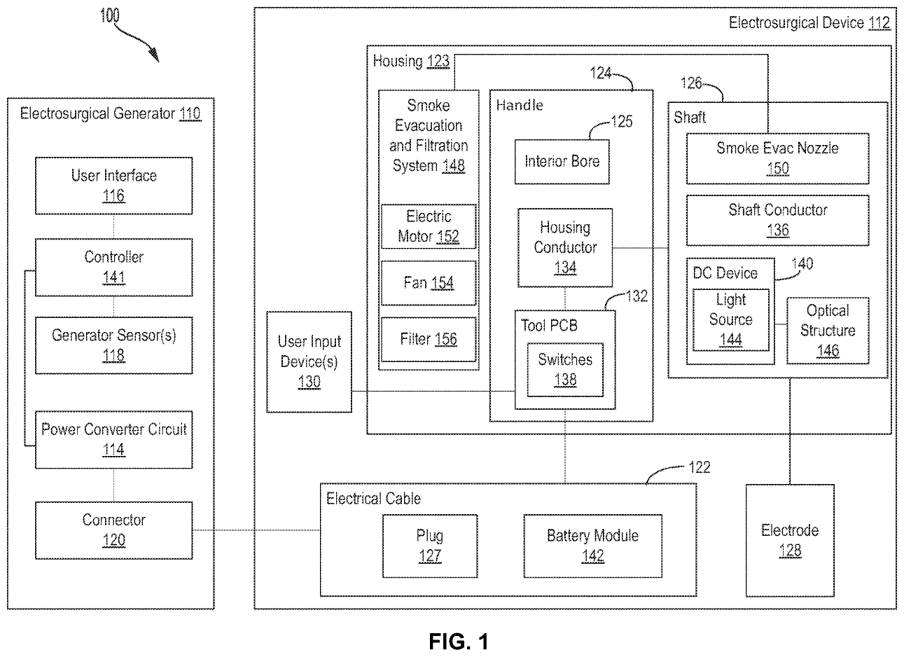

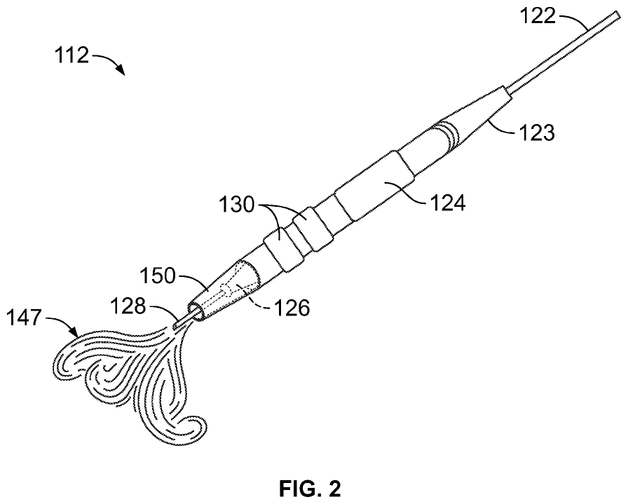

FIG. 1 illustrates an electrosurgical system 100 having an electrosurgical generator 110 and an electrosurgical device 112, in accordance with an example implementation. FIG. 2 illustrates a perspective view of the electrosurgical device 112, in accordance with an example implementation. In FIG. 2, the electrosurgical device 112 is depicted as an electrosurgical pencil as an example for illustration. FIGS. 1, 2 are described together.

In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, one or more display screens, one or more indicator lights, one or more speakers, and/or one or more haptic output devices.

In an example, the user interface 116 can be operable to select a mode of operation from among a plurality of modes of operation for the electrosurgical generator 110 based on the waveform of the electrosurgical energy. As examples, the modes of operation can include a cutting mode, a coagulating mode, an ablating mode, and/or a sealing mode, each mode having a corresponding waveform for the electrosurgical energy. As such, the electrosurgical generator 110 can generate the electrosurgical energy with a waveform selected from a plurality of waveforms based, at least in part, on the mode of operation selected using the user interface 116. Combinations of these waveforms can also be formed to create blended modes.

The electrosurgical generator 110 can also include one or more generator sensors 118 that can sense one or more conditions related to the electrosurgical energy and/or the target tissue. As examples, the generator sensor(s) 118 can include one or more current sensors, one or more voltage sensors, one or more temperature sensors, and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the generator sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 kHz and approximately 500 kHz.

In FIG. 1, the electrosurgical generator 110 also includes a connector 120 that can facilitate coupling the electrosurgical generator 110 to the electrosurgical device 112. For example, the electrosurgical device 112 can include an electrical cable 122 having a plug 127, which can be coupled to a socket of the connector 120 of the electrosurgical generator 110. In this arrangement, the electrosurgical generator 110 can supply the electrosurgical energy to the electrosurgical device 112 via the coupling between the connector 120 of the electrosurgical generator 110 and the electrical cable 122 of the electrosurgical device 112. The electrical cable 122 is described in further detail below.

The electrosurgical generator 110 can further include a controller 141 that can control operation of the electrosurgical generator 110. Within examples, the controller 141 can be implemented using hardware, software, and/or firmware. For instance, the controller 141 can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller 141, thus, can receive data and store the data in the memory as well. As shown in FIG. 1, the controller 141 can be communicatively coupled with the power converter circuit 114, the user interface 116, the generator sensor(s) 118, and/or the connector 120.

As shown in FIG. 1, the electrosurgical device 112 can include a housing 123. The housing 123 can be an elongated structure in and/or on which components of the electrosurgical device 112 can be disposed. In some examples, the housing 123 can be an integral, monolithic structure. In other examples, the housing 123 can include a plurality of structures that are coupled to each other.

In FIG. 1, the housing 123 includes a handle 124 that defines an interior bore, a shaft 126 extending in a distal direction from the handle 124, and an electrosurgical electrode 128 extending in the distal direction from the shaft 126. In general, the handle 124 can be configured to facilitate a user gripping and manipulating the electrosurgical device 112 while performing electrosurgery. For example, the handle 124 can have a shape and/or a size that can facilitate a user performing electrosurgery by manipulating the electrosurgical device 112 using a single hand. In one implementation, the handle 124 can have a shape and/or a size that facilitates the user holding the electrosurgical device 112 in a writing utensil gripping manner (e.g., the electrosurgical device 112 can be an electrosurgical pencil).

Additionally, for example, the handle 124 and/or the shaft 126 can be constructed from one or more materials that are electrical insulators (e.g., a plastic material). This can facilitate insulating the user from the electrosurgical energy flowing through the electrosurgical device 112 while performing the electrosurgery.

In some implementations, the shaft 126 can be coupled to the handle 124 in a fixed and non-moveable manner. This may simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). In one example, the handle 124 and the shaft 126 can be formed as a single, monolithic structure such that the shaft 126 and the handle 124 are fixed and non-moveable relative to each other. In another example, the handle 124 and the shaft 126 can be fixedly coupled to each other by a welding coupling, an adhesive coupling, and/or another coupling that prevents movement between the handle 124 and the shaft 126.

In other implementations, the shaft 126 can be telescopically moveable relative to the handle 124. For example, the shaft 126 can be telescopically moveable in the interior bore defined by the handle 124 to extend the shaft 126 in the distal direction and retract the shaft 126 in a proximal direction relative to the handle 124 (e.g., movable along a longitudinal axis of the electrosurgical device 112). In some examples, the electrosurgical electrode 128 can be coupled to the shaft 126 and, thus, the electrosurgical electrode 128 can move together with the shaft 126 in an axial direction along the longitudinal axis relative to the handle 124. This can provide for adjusting a length of the electrosurgical device 112, which can facilitate performing electrosurgery at a plurality of different depths within tissue (e.g., due to different anatomical shapes and/or sizes of patients) and/or at a plurality of different angles.

In some implementations, the electrosurgical electrode 128 can additionally or alternatively be rotatable about an axis of rotation that is parallel to the longitudinal axis of the electrosurgical device 112. For example, a button of the user input devices 130 can trigger rotation of the electrosurgical electrode 128.

In some examples, the electrosurgical electrode 128 can be rotatable relative to the handle 124 and the shaft 126. In other examples, the electrosurgical electrode 128 can be rotationally fixed relative to the shaft 126 such that the shaft 126 and the electrosurgical electrode 128 are rotatable together relative to the handle 124. Rotating the electrosurgical electrode 128 relative to the handle 124 can facilitate adjusting an angle of the electrosurgical electrode 128 relative to one or more user input device(s) 130 of the electrosurgical device 112. In this arrangement, a user can comfortably grip the handle 124 in a position in which their fingers can comfortably operate the user input device(s) 130 while the electrosurgical electrode 128 is set at a rotational position selected from among a plurality of rotational positions relative to the handle 124 based on, for example, a location, a size, and/or a shape of a surgical site in which the user is operating.

In one implementation, the electrosurgical electrode 128 can be rotatable by more than 360 degrees relative to the handle 124. This can improve an ease of use by allowing an operator to freely rotate the electrosurgical electrode 128 without limitation. However, in other implementations, the electrosurgical electrode 128 can be rotatable by less than or equal to 360 degrees (e.g., rotatable by 180 degrees or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement.

Although it can be beneficial to provide for rotation of the electrosurgical electrode 128 relative to the handle 124 and/or the shaft 126, the electrosurgical electrode 128 can be rotationally fixed relative to the handle 124 and the shaft 126 in some implementations. This may, for example, help to simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts).

The user input device(s) 130 can select between the modes of operation of the electrosurgical device 112 and/or the electrosurgical generator 110. For instance, in one implementation, the user input device(s) 130 can be configured to select between a cutting mode of operation and a coagulation mode of operation. Responsive to actuation of the user input device(s) 130 of the electrosurgical device 112, the electrosurgical device 112 can (i) receive the electrosurgical energy with a level of power and/or a waveform corresponding to the mode of operation selected via the user input device(s) 130, and (ii) supply the electrosurgical energy to the electrosurgical electrode 128.

In FIG. 1, the electrosurgical device 112 includes a plurality of electrical components that facilitate supplying the electrosurgical energy, which the electrosurgical device 112 receives from the electrosurgical generator 110, to the electrosurgical electrode 128. For example, the electrosurgical device 112 can include at least one electrical component selected from a group of electrical components including: a tool printed circuit board (tool PCB) 132 (e.g., a flexible printed circuit board), a housing conductor 134, and/or a shaft conductor 136 that can provide a circuit for conducting the electrosurgical energy from the electrical cable 122 to the electrosurgical electrode 128. One or more of the electrical components can be positioned in an interior bore 125 defined by the handle 124 and/or in the inner cavity defined by the shaft 126.

Within examples, the user input device(s) 130 can include one or more buttons on an exterior surface of the handle 124. Each button of the user input device(s) 130 can be operable to actuate a respective one of a plurality of switches 138 of the tool PCB 132. In general, the switches 138 and/or the tool PCB 132 are operable to control a supply of the electrosurgical energy from the electrosurgical generator 110 to the electrosurgical electrode 128. For instance, in one implementation, when each button is operated (e.g., depressed), the respective switch 138 associated with the button can be actuated to cause the tool PCB 132 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 138 associated with the button can close the switch 138 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the tool PCB 132 can be omitted.

In both example implementations, the electrosurgical energy supplied by the electrosurgical generator 110 can be supplied from (i) the electrical cable 122, the tool PCB 132, and/or the switches 138 to (ii) the electrosurgical electrode 128 by the housing conductor 134 and the shaft conductor 136. As such, as shown in FIG. 1, the tool PCB 132 can be coupled to the electrical cable 122, the housing conductor 134 can be coupled to the tool PCB 132 and the shaft conductor 136, and the shaft conductor 136 can be coupled to the electrosurgical electrode 128. In this arrangement, the housing conductor 134 can conduct the electrosurgical energy (supplied to the housing conductor 134 via the tool PCB 132) to the shaft conductor 136, and the shaft conductor 136 can conduct the electrosurgical energy to the electrosurgical electrode 128.

In general, the housing conductor 134 and the shaft conductor 136 can each include one or more electrically conductive elements that provide an electrically conductive bus for supplying the electrosurgical energy to the electrosurgical electrode 128. More particularly, the housing conductor 134 can include one or more electrically conductive elements of the handle 124 that can supply the electrosurgical energy to the shaft conductor 136, and the shaft conductor 136 can include one or more electrically conductive elements of the shaft 126 that can supply the electrical energy from the housing conductor 134 to the electrosurgical electrode 128. In implementations in which the shaft 126 is movable or rotatable relative to the handle 124, the housing conductor 134 can engage the shaft conductor 136 to maintain an electrical coupling between the housing conductor 134, the shaft conductor 136, and the electrosurgical electrode 128 while (i) the shaft 126 and/or the electrosurgical electrode 128 telescopically moves relative to the handle 124, and/or (ii) the electrosurgical electrode 128 rotates relative to the handle 124.

Although the electrosurgical device 112 includes the user input device(s) 130 in FIG. 1, the user input device(s) 130 can be separate from the electrosurgical device 112 in another example. For instance, the user input device(s) 130 can additionally or alternatively include one or more foot pedals that are actuatable to control operation of the electrosurgical device 112 as described above. The foot pedal(s) can be communicatively coupled to the electrosurgical generator 110 to provide a signal responsive to actuation of the foot pedal(s).

As noted above, the electrosurgical electrode 128 can apply the electrosurgical energy to a target tissue to perform an electrosurgical operation (e.g., cutting, coagulating, ablating, and/or sealing the target tissue). Within examples, the electrosurgical electrode 128 can include an electrosurgical substrate formed from an electrically conductive material. As an example, the electrically conductive material can be stainless steel.

The electrosurgical substrate can extend in an axial direction from a proximal end of the electrosurgical electrode 128 to a distal end of the electrosurgical electrode 128. The proximal end of the electrosurgical electrode 128 can receive electrosurgical energy (e.g., via the housing conductor 134 and the shaft conductor 136 as described above), and a distal working portion of the electrosurgical electrode 128 can apply the electrosurgical energy to the target tissue. In one implementation, the electrosurgical substrate can include a shank portion that extends from the proximal end of electrosurgical electrode 128 to the distal working portion of the electrosurgical electrode 128. The distal working portion can be configured to use the electrosurgical energy to at least one of cut or coagulate tissue in a monopolar electrosurgical operation.

In some examples, the distal working portion can define an electrosurgical blade. For instance, the electrosurgical blade can include (i) a first lateral surface, (ii) a second lateral surface opposite the first lateral surface, (iii) a first major surface extending between the first lateral surface and the second lateral surface on a first side of the electrosurgical blade, and (iv) a second major surface extending between the first lateral surface and the second lateral surface on a second side of the electrosurgical blade that is opposite the first side. The first lateral surface and the second lateral surface have surface areas that are relatively small compared to surface areas of the first major surface and the second major surface such that a thickness (e.g., a dimension between the first major surface and the second major surface) of the electrosurgical blade is relatively small as compared to a length (e.g., a dimension extending between the proximal end and the distal end of the electrosurgical electrode 128) and a width (e.g., a dimension between the first lateral surface and the second lateral surface).

In some examples, the distal working portion of the electrosurgical electrode 128 can also include an outer layer of material covering at least a portion (or an entirety) of the electrosurgical substrate. For instance, the outer layer of material can be formed from at least one material of: a polymeric material, a fluorocarbon material (e.g., polytetrafluoroethylene (PTFE)), silicone, enamel, a ceramic material, and inorganic lubricant material (e.g., titanium nitride, zirconium nitride, titanium aluminum nitride, and nitron). The outer layer of material can help to, for example, inhibit eschar build-up and/or focus the electrosurgical energy to one or more portions of the electrosurgical electrode 128.

As shown in FIG. 1, the electrosurgical device 112 includes at least one direct current (DC) device 140 and a battery module 142. In general, the DC device 140 is configured to use a DC power provided by the battery module 142 to perform a function in connection with the electrosurgical system 100. The DC device 140 can be disposed at least partially or entirely in the housing 123 and/or at least partially or entirely on an exterior surface of the housing 123. As examples, the DC device 140 can include at least one device of: one or more DC powered sensors, one or more cameras, one or more ultrasound transmitters, one or more light sources 144, one or more haptic devices, and one or more fluid pumps.

In examples that include a DC powered sensor, the DC power sensor can sense one or more operational conditions during an electrosurgical procedure. For instance, the DC powered sensor(s) can include at least one sensor of: (i) a temperature sensor, (ii) an electrochemical sensor, (iii) a force sensor, (iv) a mass loading sensor, (v) a dielectric sensor, (vi) a conductivity sensor, (vii) a metal detector sensor, (viii) a tracking sensor configured to sense at least one of: a location of the electrosurgical electrode and an orientation of the electrosurgical electrode, (ix) light sensor, and (x) a smoke detector sensor (e.g., a Volatile Organic Compounds (VOC) sensor). Within examples, the DC powered sensor(s) transmit sensor signals to the controller 141 of the electrosurgical generator 110 to provide a basis for feedback control of the electrosurgical system 100 and improve the electrosurgical procedure.

In examples that include a camera, the camera can use the DC power provided by the battery module 142 to capture an image of an area of interest. For instance, the camera can be configured to have a field of view that is directed in a distal direction to capture an image of the electrosurgical electrode 128, a target tissue, and/or a surgical site. This can help a user to visualize cutting and/or coagulating the target tissue.

In examples that include the light source(s) 144, the light source(s) 144 can generate light that can be emitted by the electrosurgical device 112 to illuminate an area of interest (e.g., a target tissue at the surgical site). In some implementations, the light source(s) 144 can be located at a distal end of the housing 123 and/or a distal end of the shaft 126 to directly provide light in a distal direction and illuminate a surgical distal of the electrosurgical electrode 128.

In other implementations, as shown in FIG. 1, the light source(s) 144 can be optically coupled to an optical structure 146, which is configured to receive the light emitted by the light source(s) 144 and transmit the light in a distal direction toward a surgical site to illuminate the surgical site while performing electrosurgery using the electrosurgical electrode 128. Although arranging the light source(s) 144 to directly illuminate a surgical field can help, for instance, to reduce a cost of manufacture, transmitting the light using the optical structure 146 can help to improve a quality of light transmitted from the electrosurgical device 112 (e.g., by providing light with improved uniformity and/or reduced heat generation).

As examples, in implementations that include the optical structure 146, the optical structure 146 can include at least one optical structure of an optical lens, a non-fiber optic optical waveguide, and an optical fiber. When the optical structure 146 includes the optical lens (e.g., a parabolic reflector lens, an aspheric lens, and/or a Fresnel lens), the optical structure 146 can help to direct the light emitted by the light source 144 in the distal direction and thereby improve a quality of the light illuminating the surgical site. The optical structure 146 can additionally or alternatively include the non-fiber optic optical waveguide and/or the optical fiber to transmit the light over relatively large distances in the shaft 126. For instance, the optical waveguide can transmit the light in the distal direction via total internal reflection. In such implementations, the optical waveguide can include a cladding and/or an air gap on an exterior surface of the optical waveguide to help facilitate total internal reflection. In some implementations, the non-fiber optic optical waveguide can be formed as a single, monolithic structure.

In some examples, the optical structure 146 can additionally or alternatively include other light shaping optical elements such as, for instance, a plurality of facets, one or more prisms, and/or one or more optical gratings. Although the optical structure 146 can help to improve a quality of the light directed to the surgical site, the electrosurgical device 112 can omit the optical structure 146 and instead emit the light from the light source 144 directly to the surgical field without transmitting the light through the optical structure 146 in other examples.

In FIG. 1, the light source 144 can be coupled to the shaft 126. As such, the light source 144 can also move telescopically with the shaft 126 relative to the handle 124. However, in other examples, the light source 144 can be in the interior bore of the handle 124 and/or coupled to an exterior surface of the handle 124. As examples, the light source 144 can include one or more light emitting diodes (LEDs), organic light emitting diodes (OLEDs), optical fibers, non-fiber optic waveguides, and/or lenses. Additionally, for example, the light source 144 can include a light-emitting diode printed circuit board (LED PCB) having one or more light sources (e.g., LEDs). The LED PCB can include a PCB aperture, and one or more other components (e.g., the electrosurgical electrode 128) of the electrosurgical device 112 can extend through the aperture.

The optical structure 146 can be at a distal end of the shaft 126. In some examples, the optical structure 146 can circumferentially surround the electrosurgical electrode 128 to emit the light distally around all sides of the electrosurgical electrode 128. This can help to mitigate shadows and provide greater uniformity of illumination in all rotational alignments of the shaft 126 relative to the housing 123 and/or the electrosurgical device 112 relative to the target tissue. However, in other examples, the optical structure 146 can extend partially but not fully around the electrosurgical electrode 128.

Within examples, the user input device(s) 130, the tool PCB 132, the switches 138, the housing conductor 134, the shaft conductor 136, the electrical cable 122, and/or the battery module 142 can supply the DC electrical power from the battery module 142 to the DC device 140.

The user input device(s) 130 can be actuated to operate the DC device(s) 140 (e.g., to cause the light source(s) 144 to emit light). In one example, the user input device(s) 130 can include a button that independently controls the DC device(s) 140 separate from the button(s) that control the electrosurgical operational modes of the electrosurgical device 112. In another example, the user input device(s) 130 and the tool PCB 132 can be configured such that operation of the button(s) that control the electrosurgical operational mode simultaneously control operation of the DC devices 140 (e.g., the light source 144 can be automatically actuated to emit light when a button is operated to apply the electrosurgical energy at the electrosurgical electrode 128).

As shown in FIG. 1, responsive to operation of the user input device(s) 130 to actuate the DC device(s) 140, the battery module 142 can supply the electrical power (e.g., a DC voltage) to the DC device(s) 140 via the electrical cable 122, the tool PCB 132, the housing conductor 134, and/or the shaft conductor 136. In this implementation, one or more of the conductive elements of the housing conductor 134 can be configured to supply the electrical power from the battery module 142 to the DC device(s) 140 and/or return the electrical power from the DC device(s) 140 to the battery module 142. Accordingly, the housing conductor 134 can additionally or alternatively assist in providing electrical communication between the battery module 142 and the DC device(s) 140 as the shaft 126 and the light source 144 telescopically move and/or rotate relative to the handle 124.

Although the user input device(s) 130 on the handle 124 can be operated to control the operation of the DC device(s) 140 in the examples described above, the DC device(s) 140 can be additionally or alternatively operated by one or more user input device(s) on the electrosurgical generator 110 (e.g., via the user interface 116) and/or on the plug 127 of the electrical cable 122)

Within examples, the electrosurgical device 112 can additionally or alternatively include features that provide for evacuating and filtering of surgical smoke 147 (shown in FIG. 2) from the distal end of the shaft 126 and/or the electrosurgical electrode 128. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, the electrosurgical device 112 includes a smoke evacuation and filtration system 148 integrated in or with the electrosurgical device 112. In FIG. 1, the smoke evacuation and filtration system 148 can be disposed in an inner cavity within the housing 123 and is shown outside the handle 124 and the shaft 126. However, it should be understood that the smoke evacuation and filtration system 148 can be disposed, at least partially, within the handle 124 and/or the shaft 126.

In an example, the electrosurgical device 112 can have a smoke evacuation nozzle 150 disposed about a portion of the shaft 126 and a portion of the electrosurgical electrode 128 (see FIG. 2). The smoke evacuation nozzle 150 extends circumferentially around a center axis of a distal portion of the electrosurgical electrode 128. In this arrangement, the smoke evacuation nozzle 150 defines a smoke inlet to receive the surgical smoke 147 into the smoke evacuation nozzle 150 in all rotational alignments of the electrosurgical electrode 128 relative to the handle 124 and/or the electrosurgical device 112. However, in another example, the smoke evacuation nozzle 150 can include one or more smoke inlets that do not extend circumferentially around the electrosurgical electrode 128.

In one example, the smoke evacuation nozzle 150 can be separate and independent from the shaft 126. The smoke evacuation nozzle 150 is configured to capture and channel the surgical smoke 147 to within the housing 123. Particularly, the smoke evacuation nozzle 150 operates as an intake or inlet for ingress of the surgical smoke 147 into the electrosurgical device 112.

The smoke evacuation and filtration system 148 comprises an electric motor 152 coupled to and configured to drive an impeller or fan 154. For example, the electric motor 152 can be a brushless DC micro-motor. Within examples, the user input device(s) 130, the tool PCB 132, the switches 138, the housing conductor 134, the shaft conductor 136, the electrical cable 122, and/or the battery module 142 can supply electrical power from the battery module 142 to the electric motor 152.

As the electric motor 152 drives the fan 154. In an example, the fan 154 is a suction micro fan configured to generate sufficient suction to draw the surgical smoke 147 through the smoke evacuation nozzle 150 into the housing 123. The smoke evacuation and filtration system 148 further comprises a filter 156 through which the smoke drawn via the fan 154 passes to separate toxic particulate matter from the surgical smoke 147.

In an example, the filter 156 includes an ultra-low particulate air (ULPA) filter (e.g., including activated carbon). In one example, the filter 156 can be duped with silver to disinfect the surgical smoke 147 from any bacteria. As such, the surgical smoke 147 is cleaned (e.g., smoke is absorbed) and disinfected via the filter 156. The cleaned air is then released to the environment of the electrosurgical device 112 (e.g., to the operating room) and is safe to inhale.

In an example, the smoke evacuation nozzle 150 defines a first portion of a smoke flow path, and the interior bore 125 of the handle 124, or the inner cavity of the shaft 126, defines a second portion of a smoke flow path. In this arrangement, the surgical smoke 147 can be received from the surgical site into the smoke evacuation nozzle 150, and flow proximally along the smoke evacuation nozzle 150 to the interior bore 125 of the handle 124 or the inner cavity of the shaft 126. In the interior bore 125 of the handle 124 of the inner cavity of the shaft 126, the smoke can further flow through the filter 156, and the filtered smoke/cleaned air is then discharged outside the electrosurgical device 112.

With this configuration of the smoke evacuation and filtration system 148 being integrated within the electrosurgical device 112, a separate smoke evacuator as in conventional systems is eliminated. Further, a separate smoke tubing is also eliminated, and strain on surgeon's hand resulting from handling multiple separate components may also be eliminated. This way, cost and complexity of the electrosurgical system 100 may be reduced. Further, risk of infection or any harm resulting from surgical smoke may be eliminated. Further, the electric motor 152 and the fan 154 being small do not generate much noise, compared to a suction pump of a conventional system.

Further, in an example, the electrosurgical device 112 may be a single use disposable device. Thus, there is no need to replace the filter 156. In conventional systems, filters and maintenance of the smoke evacuation system is costly and has to be performed periodically.

The smoke evacuation and filtration system 148 can take several forms or arrangements. Further, the location of the smoke evacuation and filtration system 148 can be at the distal end of the housing 123, at a proximal end of the housing 123, or anywhere between the distal end and the proximal end of the housing 123.

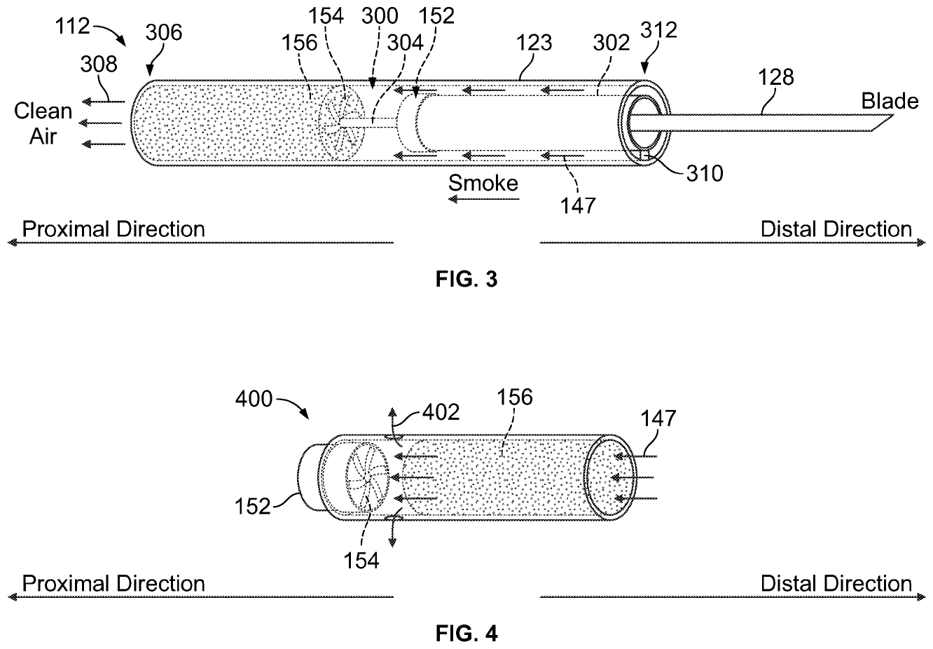

FIG. 3 illustrates a partial view of the electrosurgical device 112 with a smoke evacuation and filtration system 300, in accordance with an example implementation. The smoke evacuation and filtration system 300 represents the smoke evacuation and filtration system 148, for example. Cylinder 302 schematically represents components of the electrosurgical device 112 discussed above with respect to FIG. 1 associated with providing electric power to the electrosurgical electrode 128.

As depicted in FIG. 3, the smoke evacuation and filtration system 300 is disposed toward a proximal end 306 of the electrosurgical device 112. The electric motor 152 is disposed distal to the fan 154, which in turn is disposed distal to the filter 156. An output shaft 304 of the electric motor 152 drives the fan 154 to withdraw the surgical smoke 147 into the housing 123, and then force the surgical smoke 147 through the filter 156, which is disposed at the proximal end 306 of the electrosurgical device 112 and the housing 123.

In the implementation of FIG. 3, the filter 156 is configured as a cylinder or pod. For example, the pod can be made of activated carbon to filter VOCs from the surgical smoke 147. For instance, the filter 156 can be configured to filter the surgical smoke 147 through a bed of activated carbon (also referred to as activated charcoal) to remove VOCs from the surgical smoke 147. In an example, the filter 156 can also be duped with silver to disinfect the surgical smoke, before discharging clean air 308 from the proximal end 306 of the electrosurgical device 112 in an axial or longitudinal direction.

In one example, the operator of the electrosurgical device 112 can actuate (i.e., turn on) the electric motor 152 using the user input devices 130 (e.g., a button or the like). For example, a first user input device of the user input devices 130 can be operable to control a supply of electrosurgical energy to the electrosurgical electrode 128, and a second user input device of the user input devices 130 can be operable to actuate the electric motor 152 to rotate the fan 154 and draw the surgical smoke 147 along the smoke flow path toward the filter 156.

In one example, an input device of the user input devices 130 is used to provide or control a supply of electrosurgical energy to the electrosurgical electrode 128. For example, the input device can be a button that is pressed by a user to provide electrosurgical energy to the electrosurgical electrode 128. In this example, the electric motor 152 can be configured to be automatically actuated in response to such input device causing the electrosurgical energy to be supplied to the electrosurgical electrode. In other words, selecting (e.g., pressing) the input device causes both the electrosurgical energy to be supplied to the electrosurgical electrode 128 and the electric motor 152 to be actuated.

In another example, the electrosurgical device 112 can include a smoke sensor 310 configured to provide information related to detecting the surgical smoke 147 and/or the amount thereof. In this example, a controller of the electrosurgical device 112 (e.g., the controller 141, a processor of the tool PCB 132, or the switches 138) automatically turns the electric motor 152 on and off accordingly.

In the example implementation of FIG. 3, the smoke sensor 310 is disposed at a distal end 312 of the electrosurgical device 112 at the inlet of the surgical smoke 147 into the housing 123. However, in other example implementations, the smoke sensor 310 can be placed anywhere along a smoke path of the surgical smoke 147 within the electrosurgical device 112 (e.g., within the housing 123).

As an example, the smoke sensor 310 can be a VOC sensor configured to detect changes in specific gases in the air around the electrosurgical device 112. If the VOC sensor provides information indicating presence of the surgical smoke 147 generated as the electrosurgical electrode 128 interacts with tissues of a patient, a controller of the electrosurgical device 112 can in response automatically turn on the electric motor 152 to draw, filter, and disinfect the surgical smoke 147.

In one example, the VOC sensor can also provide information indicative of an amount of surgical smoke (e.g., the amount of specific particles in the surgical smoke 147) and the controller can turn the electric motor 152 on when the amount exceeds a threshold amount. If the VOC sensor indicates that no surgical smoke is present or that the amount of specific particles is below a threshold, the controller automatically shuts off the electric motor 152. This way, the controller is configured to control, based on the amount of the surgical smoke 147 indicated by a signal from the VOC sensor, an amount of suction generated by the electric motor 152 (e.g., control the speed of the electric motor 152) and the fan 154.

In one example, the electrosurgical device 112 can include a moisture sensor. Such moisture sensor can be comprised in the smoke sensor 310 or can be an additional sensor. The moisture sensor is configured to sense a moisture in the smoke flow path within the housing 123.

The moisture sensor can be configured to provide to the controller of the electrosurgical device 112 a moisture signal that is indicative of the moisture sensed by the moisture sensor. In an example, based on the moisture signal, the controller is configured to deactivate the electric motor 152 and stop suction of the surgical smoke 147.

In an example, the electrosurgical device 112 can further include a filter sensor that is configured to sense a parameter related to a degradation or life of the filter 156. The parameter can include, for example, an amount of suction in the smoke flow path or an amount of electrical power drawn by the electric motor 152 over a particular period. In one example, if the parameter exceeds a threshold value, the controller can provide an indication to a user of the electrosurgical device 112 (e.g., via the user interface 116) to replace the filter 156.

The implementation shown in FIG. 3 is an example for illustration. Several variations can be implemented. For example, the relative locations of the electric motor 152, the fan 154, and the filter 156 can be changed.

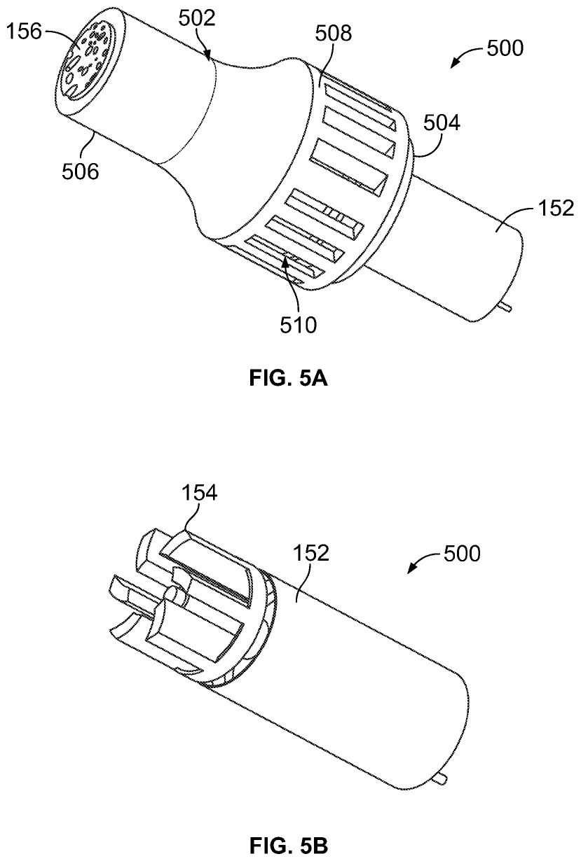

FIG. 4 illustrates a smoke evacuation and filtration system 400, in accordance with an example implementation. The smoke evacuation and filtration system 400 represents the smoke evacuation and filtration system 148, for example. The implementation of FIG. 4 differs from the implementation of FIG. 3 in that the filter 156 is disposed distal to the fan 154, which in turn is disposed distal to the electric motor 152. Further, while the surgical smoke 147 is drawn along an axial direction, clear air 402 is discharged in a radial direction as opposed to an axial direction relative to the electrosurgical device 112. The smoke evacuation and filtration system 400 can be placed at a distal or proximal end of the electrosurgical device 112, or between the distal and proximal ends of the electrosurgical device 112.

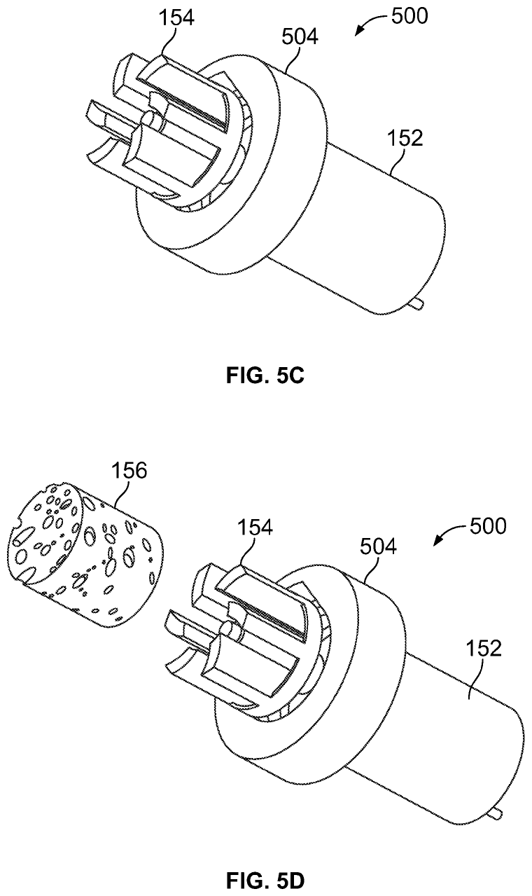

FIG. 4 represents a schematic representation of the smoke evacuation and filtration system 400. The smoke evacuation and filtration system 400 can be implemented in several ways. FIGS. 5A-5D depict one example implementation.

FIG. 5A illustrates a perspective view of a smoke evacuation and filtration system 500 having a filter housing 502, FIG. 5B illustrates a partial perspective view of the smoke evacuation and filtration system 500 showing the electric motor 152 and the fan 154, FIG. 5C illustrates another partial perspective view of the smoke evacuation and filtration system 500 showing the electric motor 152, the fan 154, and a collar 504, and FIG. 5D illustrates another partial perspective view of the smoke evacuation and filtration system 500 showing the filter 156 without the filter housing 502, in accordance with an example implementation. The smoke evacuation and filtration system 500 represents the smoke evacuation and filtration system 148 or the smoke evacuation and filtration system 400, for example. FIGS. 5A-5D are described together.

The smoke evacuation and filtration system 500 can be placed at a distal or proximal end of the electrosurgical device 112, or between the distal and proximal ends of the electrosurgical device 112. In an example, as shown in FIG. 5D, the filter 156 is disposed distal to the fan 154, which is disposed distal to the electric motor 152.

As depicted in FIG. 5A, the filter housing 502 can have a distal portion 506 that is narrow and fits over the filter 156 (or the filter 156 is inserted to the distal portion 506). The filter housing 502 is shaped as a funnel and diverges at its proximal portion 508, where the proximal portion 508 is disposed, at least partially, about or around the fan 154.

The proximal portion 508 of the filter housing 502 further has louvers 510 that are axially aligned with the fan 154. The louvers 510 are configured as axial slits disposed in a circular array about the exterior surface of the proximal portion 508 of the filter housing 502. Clean, filtered air is discharged in a radial direction through the louvers 510 as the fan 154 rotates and sucks the surgical smoke 147 through the filter 156.

In an example, the collar 504 is a hollow cylindrical component placed partially about the electric motor 152 and/or the fan 154. The collar 504 may facilitate placing the filter housing 502 about the filter 156 and may help fit the smoke evacuation and filtration system 500 as a subassembly within the electrosurgical device 112 (e.g., within the housing 123 or the shaft 126).

In an example, the smoke evacuation and filtration system 500 can be positioned at a proximal end of the housing 123 such that the louvers 510 are exposed outside the housing 123. This way, cleaned air is discharged directly to the environment of the electrosurgical device 112 through the louvers 510. In another example, the smoke evacuation and filtration system 500 can be placed within the housing 123.

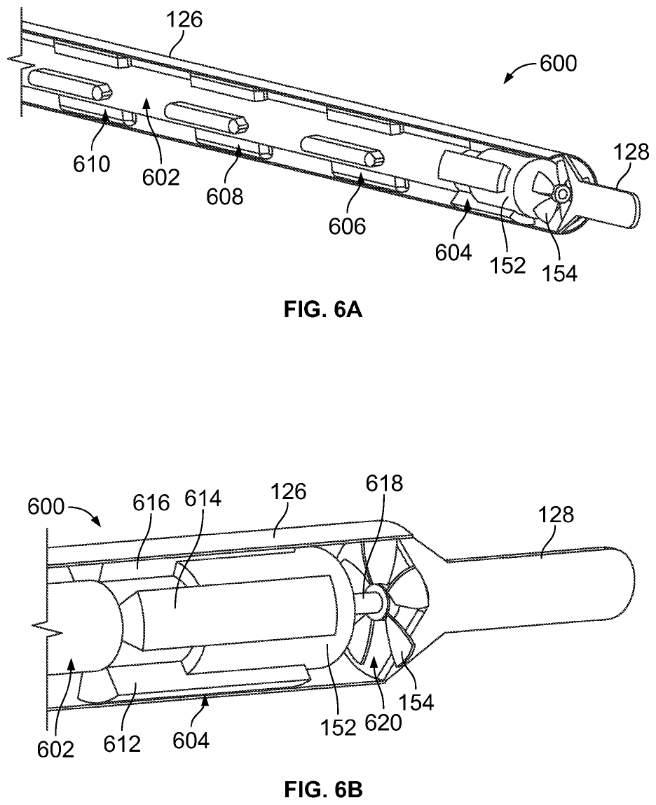

The smoke evacuation and filtration systems discussed above are positioned within the electrosurgical device 112 or coupled to the housing 123 thereof. For example, the smoke evacuation and filtration system can be placed within the housing 123 or the shaft 126. Components of the smoke evacuation and filtration system can be positioned within the housing 123 or the shaft 126 in several ways. An example mounting configuration is shown in FIGS. 6A-6B.

FIG. 6A illustrates a partial perspective view of an electrosurgical device 600, and FIG. 6B illustrates a partial perspective view of the electrosurgical device 600 from a different angle, in accordance with an example implementation. FIGS. 6A-6B provide an internal view of the shaft 126 of the electrosurgical device 600. The electrosurgical device 600 can represent the electrosurgical device 112, for example.

The shaft 126 is depicted a hollow shaft/cylinder in FIGS. 6A-6B and is coupled to the electrosurgical electrode 128. The electrosurgical device 600 includes a mounting rod 602 disposed within the shaft 126 and configured to position and center the components of the smoke evacuation and filtration system, such as the electric motor 152 and the fan 154, within the shaft 126.

The mounting rod 602 is generally cylindrical in shape. Further, the mounting rod 602 has at least one set of radial protrusions, where the radial protrusions are disposed in a circular array about the mounting rod 602. In the example implementation of FIGS. 6A-6B, the mounting rod 602 has a multiple sets of radial protrusions, and the sets of radial protrusions are axially spaced from each other along a length of the mounting rod 602. For example, the mounting rod 602 has a first set of radial protrusions 604, a second set of radial protrusions 606, a third set of radial protrusions 608, and a fourth set of radial protrusions 610. As an example, the first set of radial protrusions 604 includes four radial protrusions: radial protrusion 612, radial protrusion 614, radial protrusion 616, and a fourth radial protrusion (not shown) that is diametrically opposite from the radial protrusion 614.

The radial protrusions are disposed in circular array about the mounting rod 602, such that the exterior surfaces of the radial protrusions interface with the interior surface of the shaft 126. Further, the radial protrusions 612-616 of the first set of radial protrusions 604 have respective recessed portions to receive the electric motor 152 therein and position the electric motor 152 at the distal end of the shaft 126.

An output shaft 618 of the electric motor 152 is coupled to the fan 154 to drive the fan 154. As the fan 154 rotates, it draws the surgical smoke 147 through an aperture or opening 620 formed at the distal end of the shaft 126. The space between the radial protrusions allows the surgical smoke drawn within the shaft 126 to flow through the shaft 126.

The filter 156 is not shown in FIGS. 6A-6B to reduce visual clutter in the drawings. However, it should be understood that the filter 156 can be placed anywhere along the interior of the shaft 126 in the path of the surgical smoke suctioned via the fan 154, before being discharged from the electrosurgical device 600.

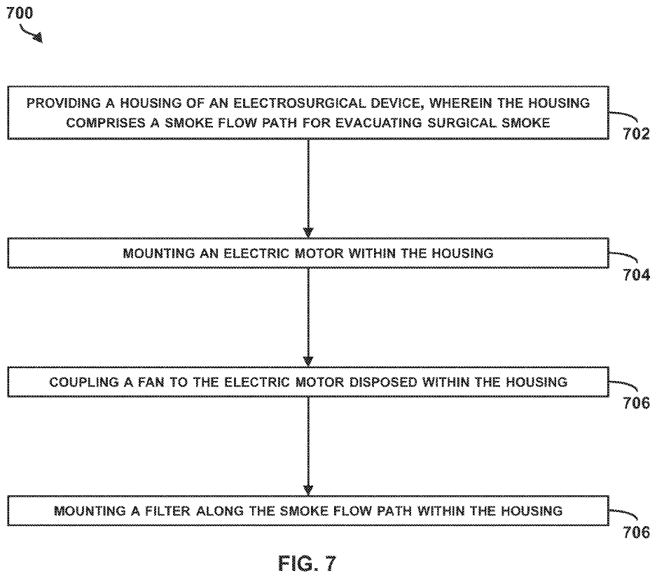

FIG. 7 is a flowchart of a method 700 of forming an electrosurgical device, in accordance with an example implementation. The electrosurgical device can be any of the surgical devices described above.

The method 700 may include one or more operations, functions, or actions as illustrated by one or more of blocks 702-708. Although the blocks are illustrated in a sequential order, these blocks may also be performed in parallel, and/or in a different order than those described herein. Also, the various blocks may be combined into fewer blocks, divided into additional blocks, and/or removed based upon the desired implementation. It should be understood that for this and other processes and methods disclosed herein, flowcharts show functionality and operation of one possible implementation of present examples. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 702, the method 700 includes providing a housing (e.g., the housing 123) of an electrosurgical device (e.g., electrosurgical device 112, 600), wherein the housing comprises a smoke flow path for evacuating surgical smoke. The term “providing” as used herein, and for example with regard to the housing 123 or other components, includes any action to make the housing 123 or any other component available for use, such as bringing the housing 123 to an apparatus or to a work environment for further processing (e.g., mounting the electric motor, fan, and filter, etc.).

At block 704, the method 700 includes mounting an electric motor (e.g., the electric motor 152), within the housing.

At block 706, the method 700 includes coupling a fan (e.g., the fan 154) to the electric motor disposed within the housing.

At block 708, the method 700 includes mounting a filter (e.g., the filter 156) along the smoke flow path within the housing.

The method 700 can further include any of the other steps or operations described throughout herein.

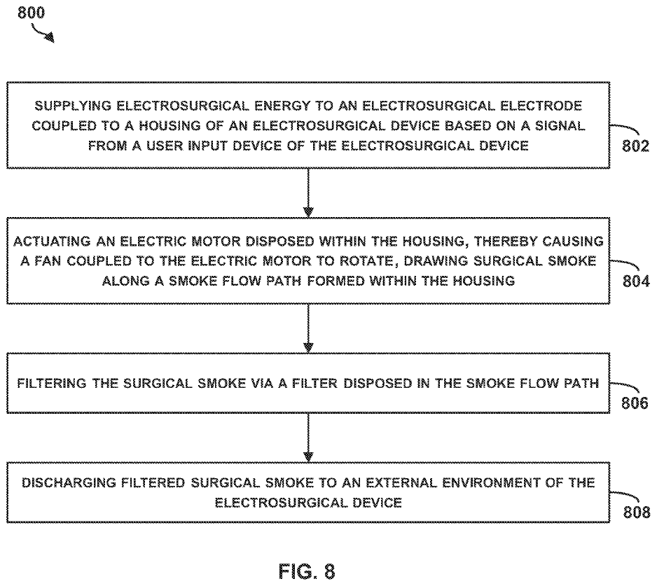

FIG. 8 is a flowchart of a method 800 of operating an electrosurgical device, in accordance with an example implementation. The electrosurgical device can be any of the surgical devices described above.

The method 800 may include one or more operations, or actions as illustrated by one or more of blocks 802-808. Although the blocks are illustrated in a sequential order, these blocks may in some instances be performed in parallel, and/or in a different order than those described herein. Also, the various blocks may be combined into fewer blocks, divided into additional blocks, and/or removed based upon the desired implementation.

In addition, for the method 800 and other processes and operations disclosed herein, the flowchart shows operation of one possible implementation of present examples. In this regard, each block may represent a module, a segment, or a portion of program code, which includes one or more instructions executable by a processor (e.g., the controller 141, a processor of the tool PCB 132, etc.) for implementing specific logical operations or steps in the process. The program code may be stored on any type of computer readable medium or memory, for example, such as a storage device including a disk or hard drive. The computer readable medium may include a non-transitory computer readable medium or memory, for example, such as computer-readable media that stores data for short periods of time like register memory, processor cache and Random Access Memory (RAM). The computer readable medium may also include non-transitory media or memory, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. In addition, for the method 800 and other processes and operations disclosed herein, one or more blocks in FIG. 8 may represent circuitry or digital logic that is arranged to perform the specific logical operations in the process.

At block 802, the method 800 includes supplying electrosurgical energy to an electrosurgical electrode (e.g., the electrosurgical electrode 128) coupled to a housing (e.g., the housing 123) of an electrosurgical device (e.g., the electrosurgical device 112, 600) based on a signal from a user input device (e.g., any of the user input device(s) 130) of the electrosurgical device.

At block 804, the method 800 includes actuating an electric motor (e.g., the electric motor 152) disposed within the housing, thereby causing a fan (e.g., the fan 154) coupled to the electric motor to rotate, drawing surgical smoke along a smoke flow path formed within the housing.

At block 806, the method 800 includes filtering the surgical smoke via a filter (e.g., the filter 156) disposed in the smoke flow path.

At block 808, the method 800 includes discharging filtered surgical smoke to an external environment of the electrosurgical device.

The method 800 can further include any of the other steps or operations described throughout herein.

The detailed description above describes various features and operations of the disclosed systems with reference to the accompanying figures. The illustrative implementations described herein are not meant to be limiting. Certain aspects of the disclosed systems can be arranged and combined in a wide variety of different configurations, all of which are contemplated herein.

Further, unless context suggests otherwise, the features illustrated in each of the figures may be used in combination with one another. Thus, the figures should be generally viewed as component aspects of one or more overall implementations, with the understanding that not all illustrated features are necessary for each implementation.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Further, devices or systems may be used or configured to perform functions presented in the figures. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner.

By the term “substantially” or “about” it is meant that the recited characteristic, parameter, or value need not be achieved exactly, but that deviations or variations, including for example, tolerances, measurement error, measurement accuracy limitations and other factors known to those skilled in the art, may occur in amounts that do not preclude the effect the characteristic was intended to provide.

The arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. Also, the terminology used herein is for the purpose of describing particular implementations only, and is not intended to be limiting.

Embodiments of the present disclosure can thus relate to one of the enumerated example embodiment (EEEs) listed below.

EEE 1 is an electrosurgical device, comprising: a housing defining therein a smoke flow path for evacuating surgical smoke; an electrosurgical electrode extending from the housing; an electric motor disposed within the housing; a fan coupled to the electric motor and disposed within the housing; and a filter disposed along the smoke flow path within the housing, wherein as the electric motor rotates the fan, the fan draws the surgical smoke to flow within the housing through the filter, then flow to an external environment of the electrosurgical device.

EEE 2 is the electrosurgical device of EEE 1, further comprising: a smoke evacuation nozzle disposed about a portion of the electrosurgical electrode, wherein the smoke evacuation nozzle defines a smoke inlet through which the surgical smoke is drawn within the housing as the fan rotates.

EEE 3 is the electrosurgical device of any of EEEs 1-2, wherein the electric motor, the fan, and the filter are disposed within the housing, such that the electric motor is distal to the fan, and the fan is distal to the filter.

EEE 4 is the electrosurgical device of any of EEEs 1-2, wherein the electric motor, the fan, and the filter are disposed within the housing, such that the filter is distal to the fan, and the fan is distal to the electric motor.

EEE 5 is the electrosurgical device of any of EEEs 1-4, wherein the electrosurgical electrode extends from a distal end of the housing, and wherein the surgical smoke is discharged in an axial direction from a proximal end of the housing after flowing through the filter.

EEE 6 is the electrosurgical device of any of EEEs 1-2, 4-5, further comprising: a filter housing coupled to the housing, wherein the filter is disposed within the filter housing distal to the fan and the electric motor.

EEE 7 is the electrosurgical device of EEE 6, wherein the filter housing is shaped as a funnel such that a proximal portion of the filter housing diverges relative to a distal portion of the filter housing, wherein the filter is disposed within the distal portion of the filter housing, and wherein the proximal portion of the filter housing is disposed, at least partially, about the fan.

EEE 8 is the electrosurgical device of EEE 7, wherein the proximal portion of the filter housing has a plurality of louvers disposed in a circular array about an exterior surface of the proximal portion of the filter housing, and wherein the plurality of louvers disposed about the fan and allow the surgical smoke to be discharged in a radial direction through the plurality of louvers.

EEE 9 is the electrosurgical device of any of EEEs 1-8, further comprising: a shaft that is hollow and disposed, at least partially, within the housing; and a mounting rod disposed within the shaft, wherein the electric motor and the fan are mounted to the mounting rod within the shaft.

EEE 10 is the electrosurgical device of EEE 9, wherein the mounting rod is generally cylindrical in shape and comprises at least one set of radial protrusions, wherein radial protrusions of the at least one set of radial protrusion are disposed in a circular array about the mounting rod, such that exterior surfaces of the radial protrusions interface with an interior surface of the shaft.

EEE 11 is the electrosurgical device of EEE 10, wherein the radial protrusions have respective recessed portions configured to receive the electric motor therein and position the electric motor at a distal end of the shaft.

EEE 12 is the electrosurgical device of any of EEEs 10-11, wherein the at least one set of radial protrusions comprises multiple sets of radial protrusions that are axially spaced from each other along a length of the mounting rod.

EEE 13 is the electrosurgical device of any of EEEs 10-12, wherein the shaft comprises an opening at a distal end of the shaft through which the surgical smoke is drawn into the shaft as the fan rotates, and wherein space between the radial protrusions allow the surgical smoke drawn within the shaft to flow through the shaft.

EEE 14 is the electrosurgical device of any of EEEs 1-13, further comprising: a plurality of user input devices on an exterior surface of the housing, wherein a first user input device of the plurality of user input devices is operable to control a supply of electrosurgical energy to the electrosurgical electrode, and wherein a second user input device of the plurality of user input devices is operable to actuate the electric motor to rotate the fan and draw the surgical smoke along the smoke flow path toward the filter.

EEE 15 is the electrosurgical device of any of EEEs 1-13, further comprising: a user input device that is operable to control a supply of electrosurgical energy to the electrosurgical electrode, wherein the electric motor is configured to be automatically actuated responsive to the user input devices causing the electrosurgical energy to be supplied to the electrosurgical electrode.

EEE 16 is the electrosurgical device of any of EEEs 1-13, further comprising: a controller; and a smoke sensor communicatively coupled to the controller, wherein the smoke sensor is configured to detect a presence of the surgical smoke and provide a signal to the controller responsive to the smoke sensor detecting the presence of the surgical smoke, and wherein the controller is configured to actuate the electric motor to rotate the fan and draw the surgical smoke along the smoke flow path and through the filter in response to the signal indicating the presence of the surgical smoke.

EEE 17 is the electrosurgical device of EEE 16, wherein the smoke sensor is further configured to detect an amount of the surgical smoke and generate the signal to indicate the amount of the surgical smoke detected by the smoke sensor, and wherein the controller is further configured to control, based on the amount of the surgical smoke indicated by the signal, an amount of suction generated by the electric motor and the fan.

EEE 18 is the electrosurgical device of any of EEEs 1-17, further comprising: a controller; and a moisture sensor communicatively coupled with the controller and configured to sense a moisture in the smoke flow path, wherein the moisture sensor is configured to provide to the controller a moisture signal that is indicative of the moisture sensed by the moisture sensor, and wherein the controller is configured to deactivate the electric motor based on the moisture signal.

EEE 19 is the electrosurgical device of any of EEEs 1-18, further comprising: a filter sensor that is configured to sense a parameter related to a degradation of the filter, wherein the parameter represents an amount of suction in the smoke flow path or an amount of electrical power drawn by the electric motor.

EEE 20 is the electrosurgical device of any of EEEs 1-19, wherein the filter is a silver doped ultra-low particulate air filter.

EEE 21 is a method of forming or assembling any of EEEs 1-20. For the example, the method of EEE 21 comprises: providing a housing of an electrosurgical device, wherein the housing comprises a smoke flow path for evacuating surgical smoke; mounting an electric motor within the housing; coupling a fan to the electric motor disposed within the housing; and mounting a filter along the smoke flow path within the housing.

EEE 22 is the method of EEE 21, further comprising: mounting an electrosurgical electrode to the housing such that the electrosurgical electrode extends from the housing; and coupling a smoke evacuation nozzle about a portion of the electrosurgical electrode, wherein the smoke evacuation nozzle defines a smoke inlet through which the surgical smoke is drawn within the housing as the fan rotates.

EEE 23 is the method of any of EEEs 21-22, wherein coupling the fan to the electric motor comprises having the electric motor distal to the fan, and wherein mounting the filter within the housing comprises having the fan distal to the filter.

EEE 24 is the method of any of EEEs 21-23, wherein coupling the fan to the electric motor comprises mounting the fan distal to the electric motor, and wherein mounting the filter within the housing comprises mounting the filter distal to the fan.

EEE 25 is the method of any of EEEs 21-24, further comprising: coupling a filter housing to the housing, wherein the filter is disposed within the filter housing distal to the fan and the electric motor.

EEE 26 is the method of EEE 25, wherein the filter housing is shaped as a funnel such that a proximal portion of the filter housing diverges relative to a distal portion of the filter housing, wherein the filter is disposed within the distal portion of the filter housing, and wherein coupling the filter housing to the housing comprises: coupling the filter housing to the housing such that the proximal portion of the filter housing is disposed, at least partially, around the fan.

EEE 27 is the method of EEE 26, wherein the proximal portion of the filter housing has a plurality of louvers disposed in a circular array about an exterior surface of the proximal portion of the filter housing, and wherein coupling the filter housing to the housing comprises: coupling the filter housing to the housing such that the plurality of louvers are disposed around the fan to allow the surgical smoke to be discharged in a radial direction through the plurality of louvers.

EEE 28 is the method of any of EEEs 21-27, further comprising: mounting a shaft, at least partially, within the housing, wherein the shaft is hollow; and positioning a mounting rod within the shaft, wherein the electric motor and the fan are mounted to the mounting rod within the shaft.

EEE 29 is the method of EEE 28, wherein the mounting rod is generally cylindrical in shape and comprises at least one set of radial protrusions, wherein radial protrusions of the at least one set of radial protrusion are disposed in a circular array about the mounting rod, such that exterior surfaces of the radial protrusions interface with an interior surface of the shaft, wherein the radial protrusions have respective recessed portions, wherein mounting the electric motor within the housing comprises: mounting the electric motor to be received in the respective recessed portions at a distal end of the shaft.

EEE 30 is a method comprising: supplying electrosurgical energy to an electrosurgical electrode coupled to a housing of an electrosurgical device based on a signal from a user input device of the electrosurgical device; actuating an electric motor disposed within the housing, thereby causing a fan coupled to the electric motor to rotate, drawing surgical smoke along a smoke flow path formed within the housing; filtering the surgical smoke via a filter disposed in the smoke flow path; and discharging filtered surgical smoke to an external environment of the electrosurgical device.

EEE 31 is the method of EEE 30, wherein the user input device is a first input device, and wherein actuating the electric motor comprises: actuating the electric motor based on a respective signal from a second user input device of the electrosurgical device.

EEE 32 is the method of any of EEEs 30-31, wherein actuating the electric motor comprises: actuating the electric motor automatically in response to supplying the electrosurgical energy to the electrosurgical electrode.

EEE 33 is the method of any of EEEs 30-32, further comprising: detecting, via a smoke sensor of the electrosurgical device, presence of the surgical smoke, wherein actuating the electric motor comprises actuating the electric motor in response to detecting the presence of the surgical smoke.

EEE 34 is the method of EEE 33, wherein the smoke sensor is configured to detect an amount of the surgical smoke and generate the signal to indicate the amount of the surgical smoke detected by the smoke sensor, and wherein actuating the electric motor comprises: controlling, based on the amount of the surgical smoke indicated by the signal, a speed of the electric motor to control an amount of suction generated by the electric motor and the fan.

EEE 35 is the method of any of EEEs 30-34, further comprising: sensing, via a moisture sensor of the electrosurgical device, moisture in the smoke flow path; and deactivating the electric motor based on sensing the moisture.

EEE 36 is the method of any of EEEs 30-35, further comprising: sensing, via a filter sensor, a parameter related to a degradation of the filter, wherein the parameter represents an amount of suction in the smoke flow path or an amount of electrical power drawn by the electric motor; and in response to the parameter exceeding a threshold value, providing an indication to replace the filter.

What is claimed is:

1. An electrosurgical device comprising:
a housing defining therein a smoke flow path for evacuating surgical smoke;
an electrosurgical electrode extending from the housing;
an electric motor disposed within the housing;
a fan coupled to the electric motor and disposed within the housing; and
a filter disposed along the smoke flow path within the housing, wherein as the electric motor rotates the fan, the fan draws the surgical smoke to flow within the housing through the filter, then flow to an external environment of the electrosurgical device.

2. The electrosurgical device of claim 1, further comprising:
a smoke evacuation nozzle disposed about a portion of the electrosurgical electrode, wherein the smoke evacuation nozzle defines a smoke inlet through which the surgical smoke is drawn within the housing as the fan rotates.

3. The electrosurgical device of claim 1, wherein the electric motor, the fan, and the filter are disposed within the housing, such that the electric motor is distal to the fan, and the fan is distal to the filter.

4. The electrosurgical device of claim 1, wherein the electric motor, the fan, and the filter are disposed within the housing, such that the filter is distal to the fan, and the fan is distal to the electric motor.

5. The electrosurgical device of claim 1, wherein the electrosurgical electrode extends from a distal end of the housing, and wherein the surgical smoke is discharged in an axial direction from a proximal end of the housing after flowing through the filter.

6. The electrosurgical device of claim 1, further comprising:
a filter housing coupled to the housing, wherein the filter is disposed within the filter housing distal to the fan and the electric motor.

7. The electrosurgical device of claim 6, wherein the filter housing is shaped as a funnel such that a proximal portion of the filter housing diverges relative to a distal portion of the filter housing, wherein the filter is disposed within the distal portion of the filter housing, and wherein the proximal portion of the filter housing is disposed, at least partially, about the fan.

8. The electrosurgical device of claim 7, wherein the proximal portion of the filter housing has a plurality of louvers disposed in a circular array about an exterior surface of the proximal portion of the filter housing, and wherein the plurality of louvers disposed about the fan and allow the surgical smoke to be discharged in a radial direction through the plurality of louvers.

9. The electrosurgical device of claim 1, further comprising:
a shaft that is hollow and disposed, at least partially, within the housing; and
a mounting rod disposed within the shaft, wherein the electric motor and the fan are mounted to the mounting rod within the shaft.

10. The electrosurgical device of claim 9, wherein the mounting rod is generally cylindrical in shape and comprises at least one set of radial protrusions, wherein radial protrusions of the at least one set of radial protrusion are disposed in a circular array about the mounting rod, such that exterior surfaces of the radial protrusions interface with an interior surface of the shaft.

11. The electrosurgical device of claim 10, wherein the radial protrusions have respective recessed portions configured to receive the electric motor therein and position the electric motor at a distal end of the shaft.

12. The electrosurgical device of claim 10, wherein the at least one set of radial protrusions comprises multiple sets of radial protrusions that are axially spaced from each other along a length of the mounting rod.

13. The electrosurgical device of claim 10, wherein the shaft comprises an opening at a distal end of the shaft through which the surgical smoke is drawn into the shaft as the fan rotates, and wherein space between the radial protrusions allow the surgical smoke drawn within the shaft to flow through the shaft.

14. The electrosurgical device of claim 1, further comprising:
a plurality of user input devices on an exterior surface of the housing, wherein a first user input device of the plurality of user input devices is operable to control a supply of electrosurgical energy to the electrosurgical electrode, and wherein a second user input device of the plurality of user input devices is operable to actuate the electric motor to rotate the fan and draw the surgical smoke along the smoke flow path toward the filter.

15. The electrosurgical device of claim 1, further comprising:
a user input device that is operable to control a supply of electrosurgical energy to the electrosurgical electrode, wherein the electric motor is configured to be automatically actuated responsive to the user input device causing the electrosurgical energy to be supplied to the electrosurgical electrode.

16. The electrosurgical device of claim 1, further comprising:
a controller; and
a smoke sensor communicatively coupled to the controller,
wherein the smoke sensor is configured to detect a presence of the surgical smoke and provide a signal to the controller responsive to the smoke sensor detecting the presence of the surgical smoke, and
wherein the controller is configured to actuate the electric motor to rotate the fan and draw the surgical smoke along the smoke flow path and through the filter in response to the signal indicating the presence of the surgical smoke.

17. The electrosurgical device of claim 16, wherein the smoke sensor is further configured to detect an amount of the surgical smoke and generate the signal to indicate the amount of the surgical smoke detected by the smoke sensor, and
wherein the controller is further configured to control, based on the amount of the surgical smoke indicated by the signal, an amount of suction generated by the electric motor and the fan.

18. The electrosurgical device of claim 1, further comprising:
a controller; and
a moisture sensor communicatively coupled with the controller and configured to sense a moisture in the smoke flow path, wherein the moisture sensor is configured to provide to the controller a moisture signal that is indicative of the moisture sensed by the moisture sensor, and wherein the controller is configured to deactivate the electric motor based on the moisture signal.

19. The electrosurgical device of claim 1, further comprising:
a filter sensor that is configured to sense a parameter related to a degradation of the filter, wherein the parameter represents an amount of suction in the smoke flow path or an amount of electrical power drawn by the electric motor.

20. The electrosurgical device of claim 1, wherein the filter is a silver doped ultra-low particulate air filter.

21. A method comprising:
providing a housing of an electrosurgical device, wherein the housing comprises a smoke flow path for evacuating surgical smoke;
mounting an electric motor within the housing;
coupling a fan to the electric motor disposed within the housing; and
mounting a filter along the smoke flow path within the housing.

22. The method of claim 21, further comprising:
mounting an electrosurgical electrode to the housing such that the electrosurgical electrode extends from the housing; and
coupling a smoke evacuation nozzle about a portion of the electrosurgical electrode, wherein the smoke evacuation nozzle defines a smoke inlet through which the surgical smoke is drawn within the housing as the fan rotates.

23. The method of claim 21, wherein coupling the fan to the electric motor comprises having the electric motor distal to the fan, and wherein mounting the filter within the housing comprises having the fan distal to the filter.

24. The method of claim 21, wherein coupling the fan to the electric motor comprises mounting the fan distal to the electric motor, and wherein mounting the filter within the housing comprises mounting the filter distal to the fan.

25. The method of claim 21, further comprising:
coupling a filter housing to the housing, wherein the filter is disposed within the filter housing distal to the fan and the electric motor.

26. The method of claim 25, wherein the filter housing is shaped as a funnel such that a proximal portion of the filter housing diverges relative to a distal portion of the filter housing, wherein the filter is disposed within the distal portion of the filter housing, and wherein coupling the filter housing to the housing comprises:
coupling the filter housing to the housing such that the proximal portion of the filter housing is disposed, at least partially, around the fan.

27. The method of claim 26, wherein the proximal portion of the filter housing has a plurality of louvers disposed in a circular array about an exterior surface of the proximal portion of the filter housing, and wherein coupling the filter housing to the housing comprises:
coupling the filter housing to the housing such that the plurality of louvers are disposed around the fan to allow the surgical smoke to be discharged in a radial direction through the plurality of louvers.

28. The method of claim 21, further comprising:
mounting a shaft, at least partially, within the housing, wherein the shaft is hollow; and
positioning a mounting rod within the shaft, wherein the electric motor and the fan are mounted to the mounting rod within the shaft.

29. The method of claim 28, wherein the mounting rod is generally cylindrical in shape and comprises at least one set of radial protrusions, wherein radial protrusions of the at least one set of radial protrusion are disposed in a circular array about the mounting rod, such that exterior surfaces of the radial protrusions interface with an interior surface of the shaft, wherein the radial protrusions have respective recessed portions, wherein mounting the electric motor within the housing comprises:
mounting the electric motor to be received in the respective recessed portions at a distal end of the shaft.

30. A method comprising:
supplying electrosurgical energy to an electrosurgical electrode coupled to a housing of an electrosurgical device based on a user input device of the electrosurgical device;
actuating an electric motor disposed within the housing, thereby causing a fan coupled to the electric motor to rotate, drawing surgical smoke along a smoke flow path formed within the housing;
filtering the surgical smoke via a filter disposed in the smoke flow path; and
discharging filtered surgical smoke to an external environment of the electrosurgical device.

31. The method of claim 30, wherein the user input device is a first input device, and wherein actuating the electric motor comprises:
actuating the electric motor based on a second user input device of the electrosurgical device.

32. The method of claim 30, wherein actuating the electric motor comprises:
actuating the electric motor automatically in response to supplying the electrosurgical energy to the electrosurgical electrode.

33. The method of claim 30, further comprising:
detecting, via a smoke sensor of the electrosurgical device, presence of the surgical smoke, wherein actuating the electric motor comprises actuating the electric motor in response to detecting the presence of the surgical smoke.

34. The method of claim 33, wherein the smoke sensor is configured to detect an amount of the surgical smoke and generate the signal to indicate the amount of the surgical smoke detected by the smoke sensor, and wherein actuating the electric motor comprises:
controlling, based on the amount of the surgical smoke indicated by the signal, a speed of the electric motor to control an amount of suction generated by the electric motor and the fan.

35. The method of claim 30, further comprising:
sensing, via a moisture sensor of the electrosurgical device, moisture in the smoke flow path; and
deactivating the electric motor based on sensing the moisture.

36. The method of claim 30, further comprising:
sensing, via a filter sensor, a parameter related to a degradation of the filter, wherein the parameter represents an amount of suction in the smoke flow path or an amount of electrical power drawn by the electric motor; and
in response to the parameter exceeding a threshold value, providing an indication to replace the filter.
